(19) United States
(12) Patent Application Publication
(10) Pub. No.: US 2023/0134238 A1
(43) Pub. Date: May 4, 2023

(54) WELL PLATE AND PETRI DISH FLUID EXCHANGE PLUG

(71) Applicant: THE REGENTS OF THE UNIVERSITY OF CALIFORNIA, Oakland, CA (US)

(72) Inventors: Peyton J. Tebon, Los Angeles, CA (US); Darin Williams, Sun Valley, CA (US)

(21) Appl. No.: 17/906,087

(22) PCT Filed: Mar. 12, 2021
(86) PCT No.: PCT/US21/22109

§ 371 Date: Sep. 12, 2022

Related U.S. Application Data
(60) Provisional application No. 62/988,840, filed on Mar. 12, 2020.

(51) Int. Cl. B01L 3/00

(52) U.S. Cl. CPC B01L 3/563; B01L 2200/026; B01L 2200/0689; B01L 2300/0829

(57) ABSTRACT

The present invention provides fluidic (both air and liquid) exchange systems configured to retrofit standard cell/tissue culture well plates (6, 12, 24, 48, 96, 384, 1536 wells, and the like) and petri dishes by plugging into the openings of each well or dish.

### STATEMENT REGARDING FEDERALLY SPONSORED RESEARCH OR DEVELOPMENT

This invention was made with government support under Grant No. 449041-AK-79854 awarded by the National Cancer Institute of the National Institutes of Health. The government has certain rights in the invention.

### BACKGROUND OF THE INVENTION

Existing well plates and petri dishes require systems for improved environmental control over internal conditions without the need for specialized equipment, engineering, or manufacturing.

Thus, there is a need in the art for improved systems for establishing fluidic exchange systems in multi-well plates and petri dishes. This invention satisfies this unmet need.

### SUMMARY OF THE INVENTION

In one aspect, the present invention provides a fluid exchange plug device configured for insertion into two or more wells of a multi-well plate, comprising: a plug body having an upper surface and a lower surface; two or more sockets positioned within the plug body, each comprising a first opening, a second opening and a lumen therebetween, wherein two or more sockets, are accessible by the first opening on the upper surface of the plug body; two or more stoppers extending from the lower surface of the plug body having a first end and a second end, wherein the second end comprises at least one opening; wherein each stopper is positioned below each socket and is sized to fit within a well of a multi-well plate, wherein the second opening is fluidly connected to the at least one opening of the second end by at least one socket lumen extending through the plug body, and wherein the second end of a first stopper is fluidly connected to the second end of at least one second stopper by a stopper lumen extending through the plug body. In one embodiment, the two or more stoppers further comprise a middle section positioned anywhere along the length of each stopper, wherein the middle section has a diameter smaller than a diameter of the two or more stoppers. In one embodiment, the two or more stoppers each comprise a gasket or O-ring positioned on the middle section. In one embodiment, the two or more stoppers each extend from the lower surface of the plug body by a height ranging between about 1-25 mm. In one embodiment, the two or more sockets each comprise a threaded interior. In one embodiment, the two or more sockets each are sized to receive fluidic or microfluidic tubing. In one embodiment, the two or more sockets are each adjacent to each other and the two or more stoppers are each adjacent to each other. In one embodiment, the plug is configured for insertion into a multi-well plate selected from the group consisting of: a 6 well plate, a 12 well plate, a 24 well plate, a 48 well plate, a 96 well plate, a 384 well plate, and a 1536 well plate. In one embodiment, the device is a single use device. In one embodiment, the device is a multiple use device.

In another aspect, the present invention provides a fluid exchange plug device configured for insertion into a petri dish, comprising: a plug body having an upper surface and a lower surface; two or more sockets positioned within the plug body, each socket having a first opening, a second opening and a lumen therebetween, wherein two or more sockets are accessible by the first opening on the upper surface of the plug body; a stopper extending from the lower surface of the plug body having a first end and a second end, wherein the second end comprises at least one opening and wherein the stopper is sized to fit within a petri dish; and wherein the second opening of each socket is fluidly connected to the at least one opening of the second end by at least one socket lumen extending through the plug body. In one embodiment, the two or more stoppers further comprise a middle section positioned anywhere along the length of the stopper, wherein the middle section has a diameter smaller than a diameter of the stopper. In one embodiment, the stopper comprises a gasket or O-ring positioned on the middle section. In one embodiment, the stopper extends from the lower surface of the plug body by a height ranging between about 1-40 mm. In one embodiment, the device further comprising at least one channel embedded in the second end of the stopper. In one embodiment, the stopper is sized to fit flush against a lower surface of a petri dish. In one embodiment, the two or more sockets each comprise a threaded interior. In one embodiment, the two or more sockets each are sized to receive fluidic or microfluidic tubing. In one embodiment, the device is a single use device. In one embodiment, the device is a multiple use device.

### BRIEF DESCRIPTION OF THE DRAWINGS

The following detailed description of invention will be better understood when read in conjunction with the appended drawings. It should be understood, however, that the invention is not limited to the precise arrangements and instrumentalities of the embodiments shown in the drawings.

FIG. 1 depicts a side view of an exemplary fluid exchange plug device of the present invention.

FIG. 2 depicts a top view of an exemplary fluid exchange plug device of the present invention.

FIG. 3 depicts a top view of an exemplary fluid exchange plug device of the present invention.

FIG. 4 depicts a top view of an exemplary fluid exchange plug device of the present invention.

FIG. 5 comprising FIG. 5A through FIG. 5E depicts an overviews of the fluid exchange plug device. FIG. 5A depicts a close-up view of a plug designed to connect two, neighboring wells in a commercially available 96 well plate. The holes and slits are specialized features required to fill the well due to the small size of this particular size (plug diameter 7 mm, channel diameter 0.5 mm). FIG. 5B depicts an overview of a large plug (size of a lid) designed to connect adjacent wells across an entire 96 well-plate. FIG. 5C depicts a reverse side of the device showing threaded holes used to attach microfluidic tubing. FIG. 5D depicts a fluid exchange plug (lid size) applied to a standard 96 well plate with a quarter section of the bottom quarter showing the microfluidic channels embedded in the device to connect arbitrary (in this case adjacent) wells. FIG. 5E depicts a close up section of the at least one socket lumen and at least one stopper lumen through the fluidic exchange plug (lid size).

FIG. 6 comprising FIG. 6A through FIG. 6B depicts a fluid exchange plug designed for a 100 mm Petri dish with two sockets. FIG. 6A depicts an isometric view showing the plug and a rubber gasket (black). FIG. 6B depicts a cross-sectional view showing the presence of fluidic channels within the device, as well as two sockets (an inlet and outlet port).

FIG. 7 comprising FIG. 7A through FIG. 7C depicts FIG. 1C depicts a fluid exchange plug designed for a 100 mm Petri dish with two sockets. FIG. 7A depicts a cross sectional view of plug applied to a commercial 100 mm well plate. The void in the plate would be filled with fluid and flow can be directed across the plate. FIG. 7B depicts a sectional view of a fluid exchange plug in a petri dish. The petri dish is shown in light blue. The plug has multiple inlets and outlets. Also, a channel geometry is manufactured into the bottom of the plug to create a parallel plate flow chamber (red) with additional ports for fluid monitoring. FIG. 7C depicts a perspective view of a half-section showing the location of the channels relative to the bottom of the petri dish.

FIG. 8 depicts multiple views of an exemplary fluid exchange plug device of the present invention.

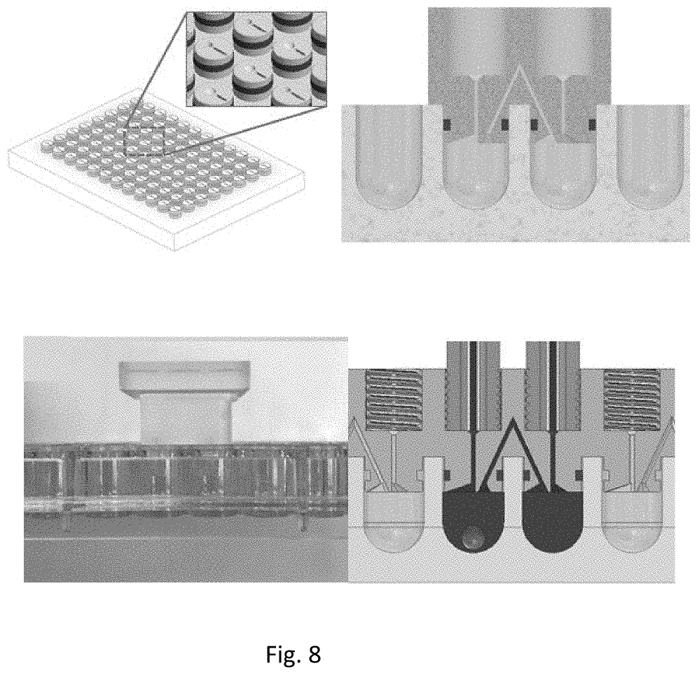

FIG. 9 depicts a side view of an exemplary well turned into perfusion bioreactors.

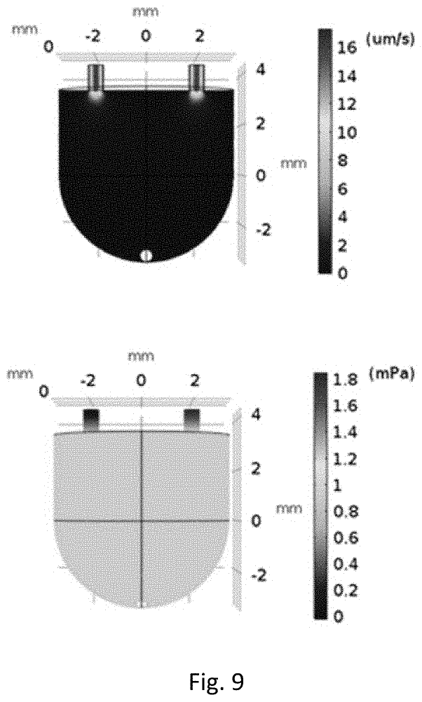

FIG. 10 depicts an exemplary implementation of the fluid exchange plug device of the present invention for the co-culture of patient-derived cell types including tumor cells and peripheral blood mononuclear cells (PBMCs). In this exemplary implementation, the fluid exchange plug device of the present invention is applied to a 96 well-plate filled with tumor spheroids. The invention allows non-invasive monitoring of the biological samples by imaging on an inverted microscope and analysis of the effluent from the well-plate chamber.

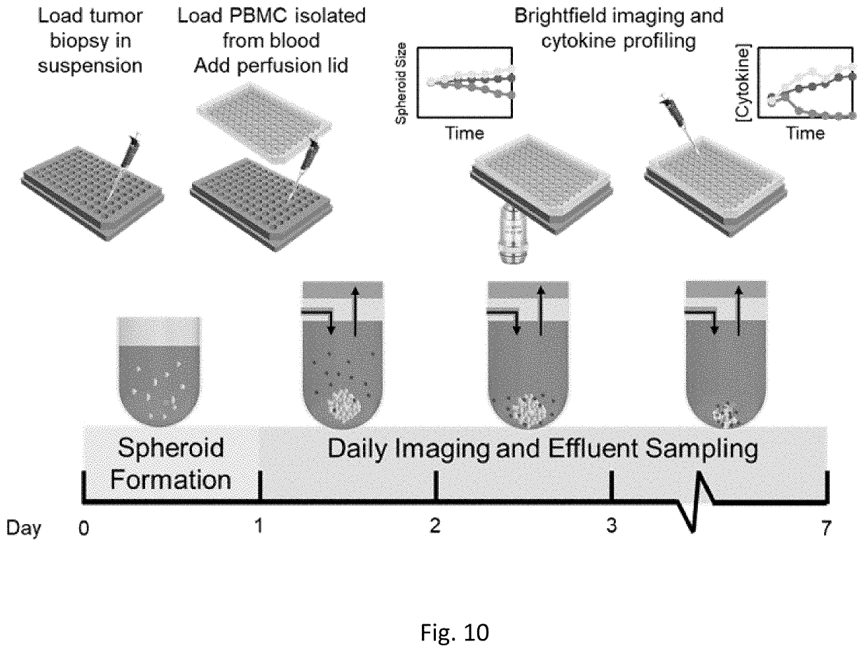

FIG. 11 depicts a top view and a side view of an exemplary fluid exchange plug device of the present invention.

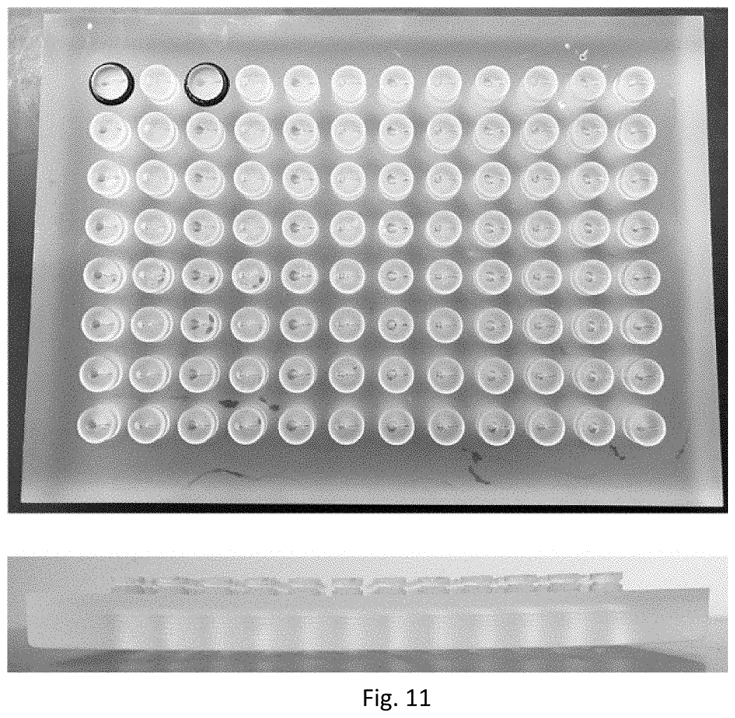

FIG. 12 depicts a side view of an exemplary fluid exchange plug device of the present invention with added tubing as inlet and an outlet.

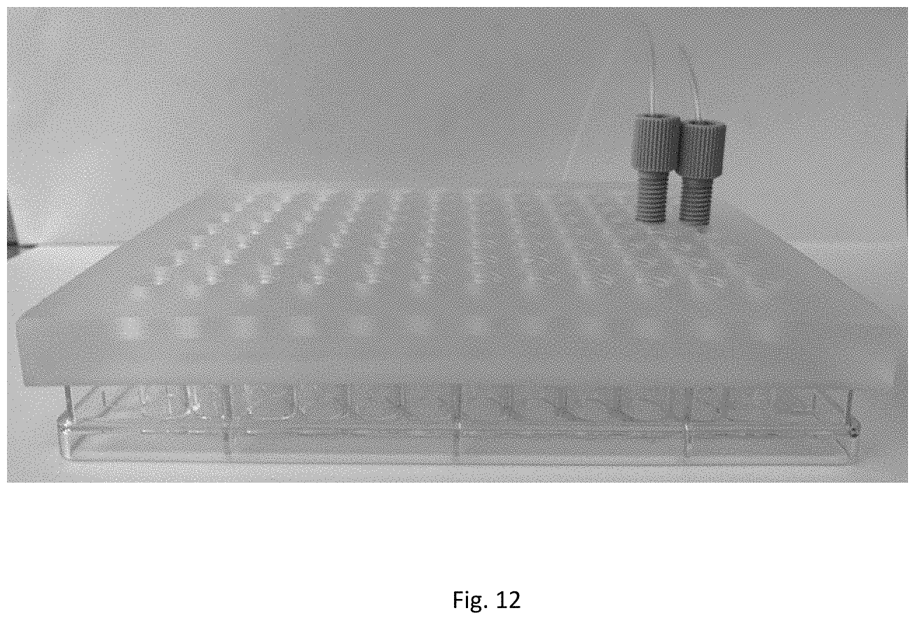

FIG. 13 depicts a top view and a perspective view of an exemplary fluid exchange plug device of the present invention.

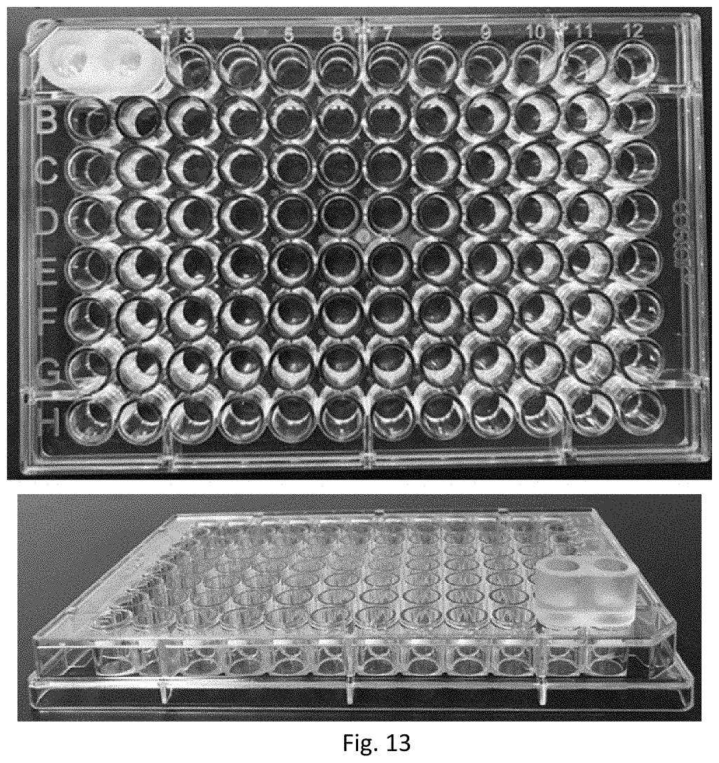

FIG. 14 depicts a top view of an exemplary fluid exchange plug device of the present invention with added tubing as inlet and an outlet.

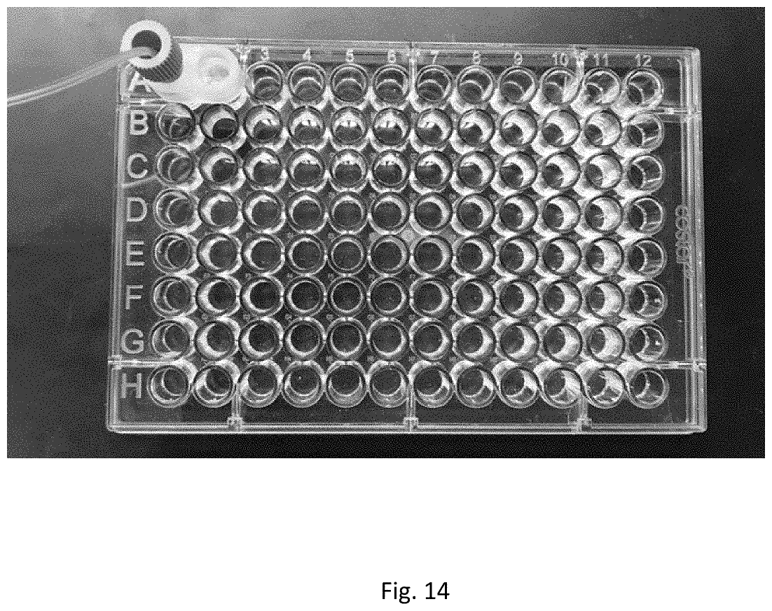

FIG. 15 depicts a close up view of an exemplary fluid exchange plug device of the present invention with added tubing as inlet and an outlet.

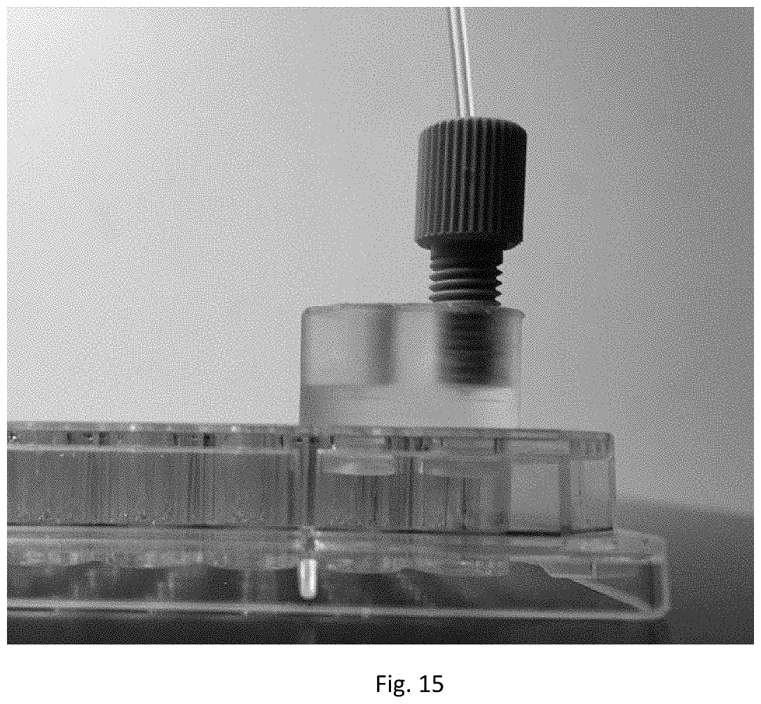

### DETAILED DESCRIPTION

### Definitions

It is to be understood that the figures and descriptions of the present invention have been simplified to illustrate elements that are relevant for a clear understanding of the present invention, while eliminating, for the purpose of clarity, many other elements typically found in the art. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Well Plate and Petri Dish Fluid Exchange Plug

The present invention provides a well plate and petri dish fluid exchange plug device configured to extend the utility of current standard well plates and petri dishes to allow improved environmental control over the internal conditions. In one embodiment, the fluid exchange plug of the present invention is configured to retrofit standard cell/tissue culture well-plates including but not limited to 6, 12, 24, 48, 96, 384, 1536 well-plate, and the like and petri dishes. In one embodiment, the fluid exchange plug of the present invention may be used for transfer of both air and liquid. In one embodiment, the fluid exchange plug device of the present invention houses fluidic channels that can be used to add, remove, and exchange fluid from well-plate and petri dish configurations in order to alter the physical, chemical, or biological conditions within the well-plate/petri dish without requiring the design of custom chemical or biological reactors. In one embodiment, the fluid exchange plug device of the present invention facilitates intermittent and continuous fluid exchange and/or perfusion through both individual wells and connected series of wells/dishes that can be used to expand the utility of current commercial well-plates and petri dishes by introducing a novel dimension of experimental conditions including but not limited to shear stress, nutrient concentration, cell-based stimuli, gas compositions and etc. In one embodiment, the device of the present invention allows for imaging during use, eliminating the need to remove the cells/samples from the environmental conditions created within the sealed compartments for imaging. In some embodiments, the device of present invention enables perfusion of biological cellular aggregates in commercially available plates. In one embodiment, the device of present invention maintains the physical form and layout of the commercially available plastics, while also adding the capabilities of custom bioreactors and fluidic systems. In one embodiment, the fluid exchange plug device of the present invention may be used to gently perfuse cell culture media into a well-plate to nourish, but not disrupt, fragile cell aggregates. In one embodiment, the fluid exchange plug device of the present invention may be used with imaging systems including but not limited to a microscope, a plate reader, a cytometer, etc.

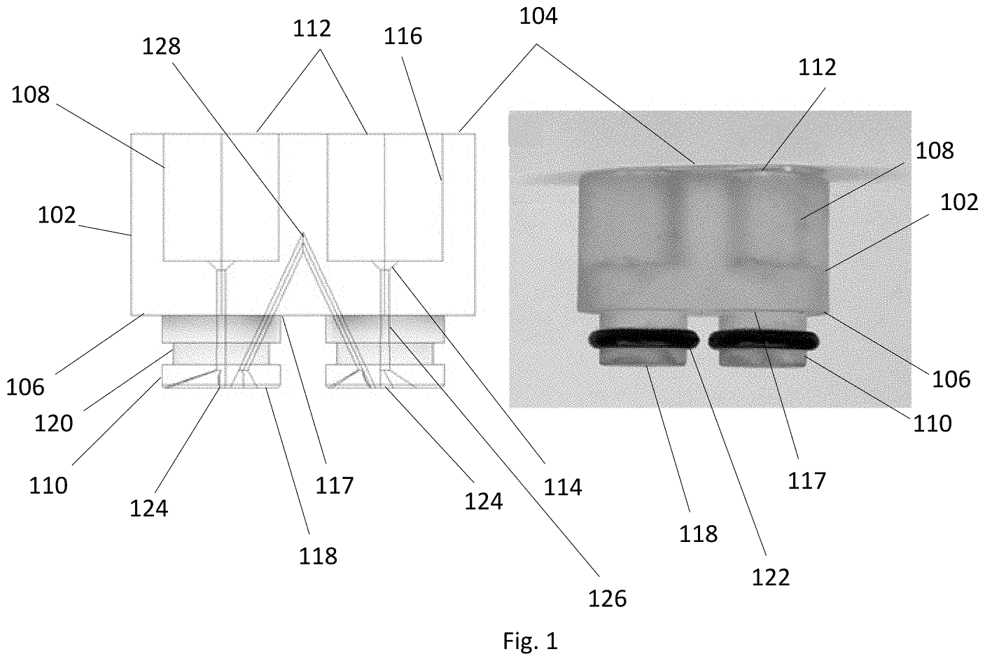

Referring now to FIG. 1, an exemplary fluid exchange plug device 100 of the present invention is shown. Fluid exchange plug 100 comprises a plug body 102 having an upper surface 104, a lower surface 106 and two or more sockets 108 positioned within plug body 102, and two or more stoppers 110.

In one embodiment, plug body 102 may have a height ranging between about 5-25 mm. In one embodiment, plug body 102 may be any shape including but not limited to circular, oval, square, rectangular, etc. In one embodiment, plug body 102 may be made of any material known to one skilled in the art including but not limited to plastics, metals, etc. In one embodiment, plug body 102 may be made of plastics such as PMMA, Acetal, polystyrene, etc. In one embodiment, plug body 102 may be made with any other plastic known to one skilled in the art. In one embodiment, plug body 102 may be made with any process known to one skilled in the art including but not limited to injection molding, milling, three-dimensional printing, etc.

Two or more sockets 108, each comprise a first opening 112, a second opening 114 and a lumen 116 therebetween. First opening 112 is positioned on upper surface 104. In one embodiment, first opening 112 has a diameter ranging between about 2-10 mm. In one embodiment, lumen 116 has a height ranging between about 2-25 mm. In one embodiment, lumen 116 may comprise a threaded interior. In one embodiment, lumen 116 may be compatible with luer locks. In one embodiment, lumen 116 may be compatible with press-in adapters. In one embodiment, two or more sockets 108 are configured to receive microfluidic tubing. In one embodiment, the two or more sockets 108 are each adjacent to each other. In one embodiment, the distance between two sockets 108 may be ranging between about 5-200 mm.

Two or more stoppers 110 are positioned in a downward direction from lower surface 106 and comprise a first end 117 and a second end 118. In one embodiment, each stopper 110 is positioned beneath each socket 108. In one embodiment, two or more stoppers 110 have a diameter sized to fit within a well of a multi-well plate. In one embodiment, multi-well plate may be selected from one including but not limited to a 6 well-plate, a 12 well-plate, a 24 well-plate, a 48 well-plate, a 96 well-plate, a 384 well-plate, a 1536 well-plate, etc. In one embodiment, two or more stoppers 110 have a height ranging between about 1-25 mm. It is understood that the dimensions of two more stoppers 110 may vary upon the dimension of the well to be sealed, and that stoppers 110 of different heights may be provided. In one embodiment, two or more stoppers 110 may enter the wells to an arbitrary depth that can be modified to be tailored to the application.

In one embodiment, two or more stoppers 110 may further have a middle section 120. In one embodiment, middle section 120 may be positioned anywhere along the length of each stopper 110. In one embodiment, middle section 120 may have a diameter smaller than the diameter of stopper 110. In one embodiment, middle section 120 is configured to receive a gasket or an O-ring 122. In one embodiment, gasket or O-ring 122 are configured to facilitate a watertight seal between fluid two or more stoppers 110 and the multi-well plates. In another embodiment, gasket or O-ring 122 may facilitate the pressurization of each well, allowing it to be used as a reservoir for downstream fluidic and microfluidic applications. In one embodiment, middle section 120 may be positioned anywhere along the length of each stopper 110. In one embodiment, gasket or O-ring 122 may facilitate the pressurization of each well, allowing it to be used for experiments requiring pressurization of a reaction or biological sample including but not limited to flowing inert gas over a chemical reaction or cyclically applying pressure to lung organoids. In one embodiment, gasket or O-ring 122 may be made from any material including but not limited to rubber.

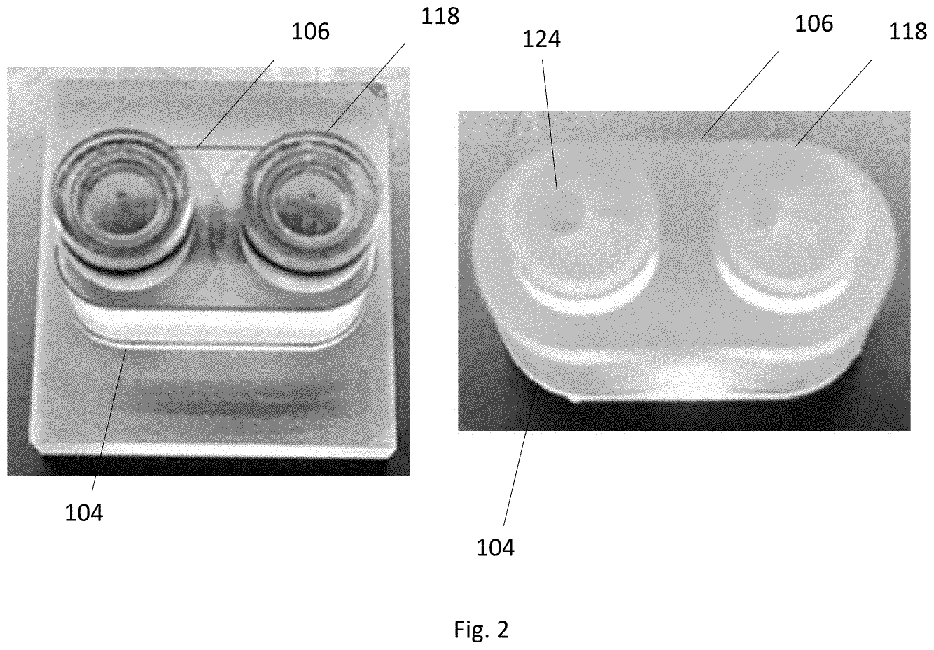

Referring now to FIG. 2, second end 118 comprises at least one opening 124. In one embodiment, at least one opening 124 may have any shape known to one in the art including but not limited to a circular, oval, square, rectangular, etc. In one embodiment, at least one opening 124 may be circular. In one embodiment, at least one opening 124 may have a diameter ranging between about 50 μm-1 cm. In one embodiment, at least one opening 124 may be a slit.

Second opening 114 is fluidly connected to at least one opening 124 through at least one socket lumen 126. In one embodiment, at least one socket lumen 126 has a diameter ranging between about 50 μm-1 cm. In one embodiment, at least one socket lumen 126 may be used to connect to fluidic tubing and external pumps and reservoir. In one embodiment, at least one socket lumen 126 may act as an inlet port configured to permit adding or supplementing culture medium to the wells. In one embodiment, at least one socket lumen 126 may act as an outlet port configured to permit removal or aspiration of culture medium from the wells. In one embodiment, fluid exchange plug device 100 may comprise at least one inlet port and at least one outlet port.

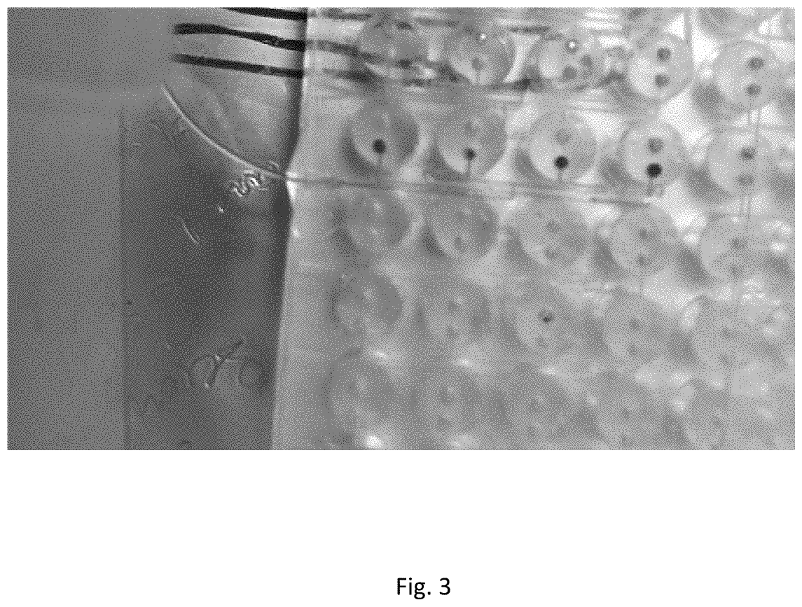

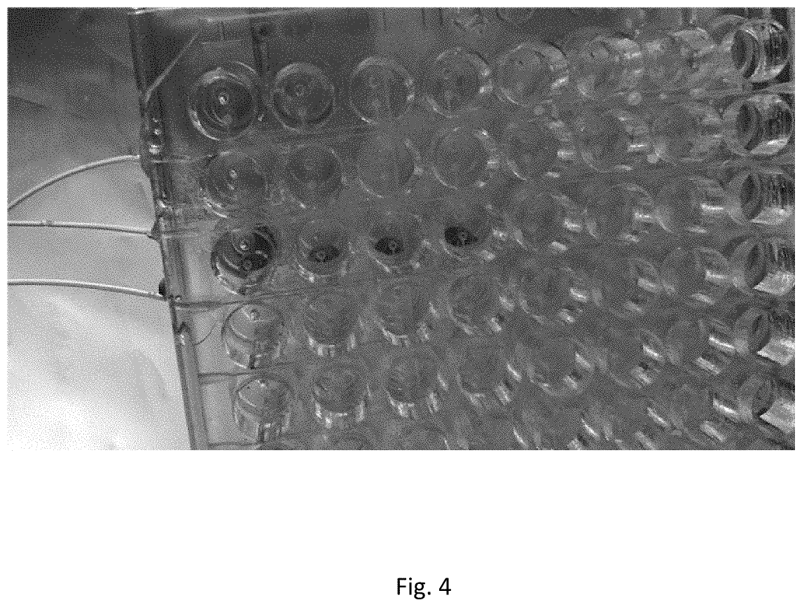

Second end 118 of two adjacent stoppers 110 may be fluidly connected to each other through a stopper lumen 128. Stopper lumen 128 extends through plug body 102, connecting two adjacent stoppers 110. In one embodiment, stopper lumen 128 may extend through plug body 102 and connect at least two stoppers 110 (FIG. 3 and FIG. 4). In one embodiment, stopper lumen 128 may connect any two stoppers together. In one embodiment, stopper lumen 128 may have a diameter ranging between about 5 μm-5 mm.

In one embodiment, fluid exchange plug device 100 may be a single use device. In one embodiment, fluid exchange plug device 100 may be a multiple use device. In one embodiment, the single or multiple use of device 100 may be configured based on the composing material.

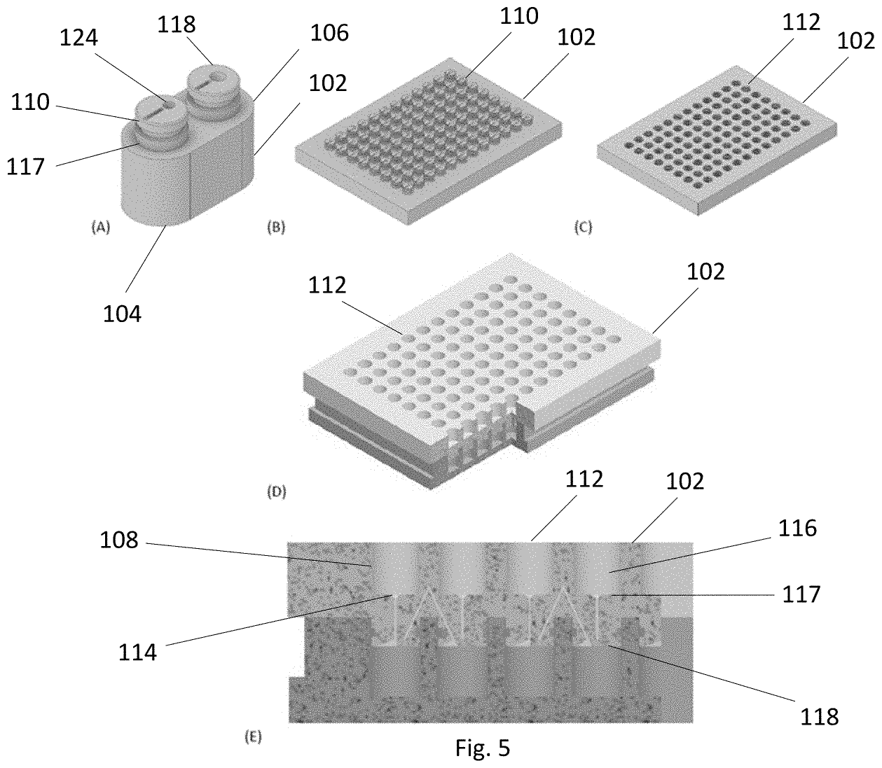

Referring now to FIG. 5A through FIG. 5E, in one embodiment, plug body 102 may comprise any number of sockets 108 and stoppers 110, enough to fill all wells of a multi-well plate.

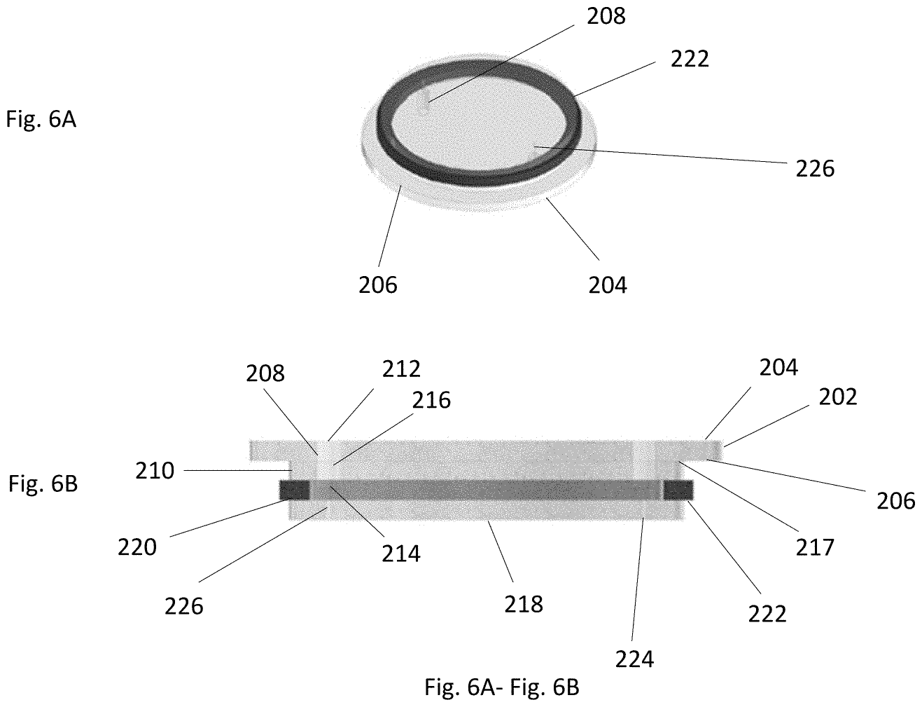

Referring now to FIG. 6A through FIG. 6B, an exemplary fluid exchange plug device 200 of the present invention is shown. Fluid exchange plug device 200 comprises a plug body 202 having an upper surface 204, a lower surface 206 and two or more sockets 208 positioned within plug body 202, and a stopper 210.

In one embodiment, plug body 202 may have a height ranging between about 5-25 mm. In one embodiment, plug body 202 may be any shape including but not limited to circular, oval, square, rectangular, etc. In one embodiment, plug body 202 may be made of any material known to one skilled in the art including but not limited to plastics, metals, etc. In one embodiment, metals may be used as to facilitate use with harsh chemicals with glass well-plates. In one embodiment, plug body 202 may be made of plastics such as PMMA, Acetal, polystyrene, etc. In one embodiment, plug body 202 may be made with any other plastic known to one skilled in the art. In one embodiment, plug body 202 may be made with any process known to one skilled in the art including but not limited to injection molding, milling, three-dimensional printing, etc.

Two or more sockets 208, each comprise a first opening 212, a second opening 214 and a lumen 216 therebetween. First opening 212 is positioned on upper surface 204. In one embodiment, first opening 212 has a diameter ranging between about 2-15 mm. In one embodiment, lumen 216 has a height ranging between about 2-25 mm. In one embodiment, lumen 216 may comprise a threaded interior. In one embodiment, lumen 216 may be compatible with luer locks. In one embodiment, lumen 216 may be compatible with press-in adapters. In one embodiment, two or more sockets 208 are configured to receive microfluidic tubing. In one embodiment, the two or more sockets 208 may be placed anywhere on lower surface 206. In one embodiment, the distance between two sockets 208 may be ranging between about 1-20 cm.

Stopper 210 is positioned in a downward direction from lower surface 206 and comprises a first end 217 and a second end 218. In one embodiment, stopper 210 is positioned beneath plug body 202. In one embodiment, stopper 210 has a diameter sized to fit within a petri dish. In one embodiment, petri dish may be selected from one including but not limited to a petri dish having a diameter of 60 mm, 100 mm, 150 mm, etc. In one embodiment, stopper 210 may have a height ranging between about 1-20 mm. It is understood that the dimensions of stopper 210 may vary upon the dimension of the petri dish to be sealed, and that stopper 210 of different heights may be provided.

In one embodiment, stopper 210 may further have a middle section 220. In one embodiment, middle section 220 may be positioned anywhere along the length of each stopper 210. In one embodiment, middle section 220 may have a diameter smaller than the diameter of stopper 210. In one embodiment, middle section 220 may have a diameter ranging between about 15-100 mm. In one embodiment, middle section 220 is configured to receive a gasket or an O-ring 222. In one embodiment, gasket or O-ring 222 are configured to facilitate a watertight seal between stopper 210 and the petri dish. In another embodiment, gasket or O-ring 222 may facilitate the pressurization of a petri dish, allowing it to be used as a reservoir for downstream fluidic and microfluidic applications. In one embodiment, gasket or O-ring 222 may facilitate the pressurization of a petri dish, allowing it to be used for experiments requiring pressurization of a reaction or biological sample including but not limited to flowing inert gas over a chemical reaction or cyclically applying pressure to lung organoids. In one embodiment, gasket or O-ring 222 may be made from any material including but not limited to rubber.

Second end 218 comprises at least one opening 224. In one embodiment, at least one opening 224 may have any shape known to one in the art including but not limited to a circular, oval, square, rectangular, etc. In one embodiment, at least one opening 224 may be circular. In one embodiment, at least one opening 224 may have a diameter ranging between about 10 μm-1 cm. In one embodiment, at least one opening 224 may be a slit.

Second opening 214 is fluidly connected to at least one opening 224 through at least one socket lumen 226. In one embodiment, at least one socket lumen 226 has a diameter ranging between about 10 μm-1 cm. In one embodiment, at least one socket lumen 226 may be used to connect to fluidic tubing and external pumps and reservoir. In one embodiment, at least one socket lumen 226 may act as an inlet port configured to permit adding or supplementing culture medium to the wells. In one embodiment, at least one socket lumen 226 may act as an outlet port configured to permit removal or aspiration of culture medium from the wells. In one embodiment, fluid exchange plug device 200 may comprise at least one inlet port and at least one outlet port.

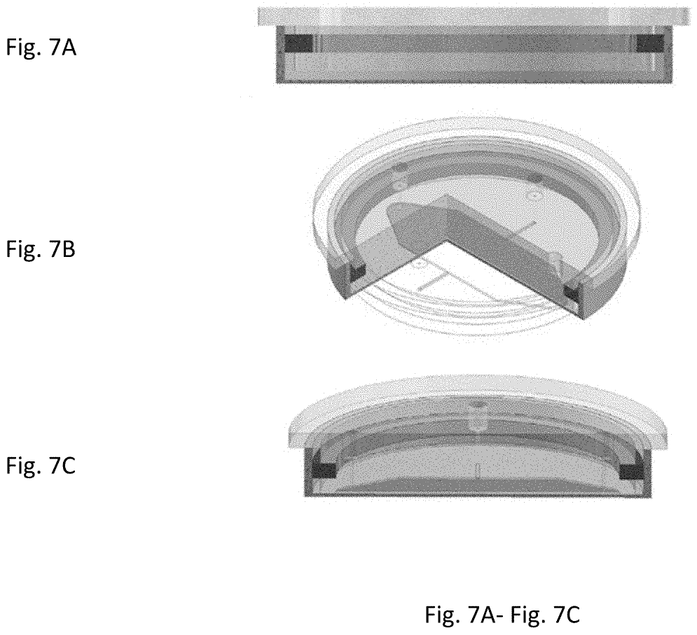

Referring now to FIG. 7A through FIG. 7C, in one embodiment, stopper 210 may comprise at least one channel 228 embedded in second end 218, wherein stopper 210 is sized to fit flush against a lower surface of a petri dish. In one embodiment, stopper 210 may have a height of ranging between about 1-40 mm to fit flush against the lower surface of the petri dish. In one embodiment, at least one channel 228 may be any shape known to one skilled in the art. In one embodiment, the geometry of at least one channel 228 may be modified to made it to adapt for the creation of fluidic and microfluidic circuits and linear flow chambers within the petri dish. In one embodiment, two or more sockets 208 may be positioned within at least one channel 228.

In one embodiment, fluid exchange plug device 200 may be a single use device. In one embodiment, fluid exchange plug device 200 may be a multiple use device. In one embodiment, the single or multiple use of device 200 may be configured based on the composing material.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A fluid exchange plug device configured for insertion into two or more wells of a multi-well plate, comprising:
a plug body having an upper surface and a lower surface;
two or more sockets positioned within the plug body, each comprising a first opening, a second opening and a lumen therebetween, wherein two or more sockets, are accessible by the first opening on the upper surface of the plug body;
two or more stoppers extending from the lower surface of the plug body having a first end and a second end, wherein the second end comprises at least one opening;
wherein each stopper is positioned below each socket and is sized to fit within a well of a multi-well plate, wherein the second opening is fluidly connected to the at least one opening of the second end by at least one socket lumen extending through the plug body, and wherein the second end of a first stopper is fluidly connected to the second end of at least one second stopper by a stopper lumen extending through the plug body.

2. The device of claim 1, wherein the two or more stoppers further comprise a middle section positioned anywhere along the length of each stopper, wherein the middle section has a diameter smaller than a diameter of the two or more stoppers.

3. The device of claim 2, wherein the two or more stoppers each comprise a gasket or O-ring positioned on the middle section.

4. The device of claim 1, wherein the two or more stoppers each extend from the lower surface of the plug body by a height ranging between about 1-25 mm.

5. The device of claim 1, wherein the two or more sockets each comprise a threaded interior.

6. The device of claim 1, wherein the two or more sockets each are sized to receive fluidic or microfluidic tubing.

7. The device of claim 1, wherein the two or more sockets are each adjacent to each other and the two or more stoppers are each adjacent to each other.

8. The device of claim 1, wherein the plug is configured for insertion into a multi-well plate selected from the group consisting of: a 6 well plate, a 12 well plate, a 24 well plate, a 48 well plate, a 96 well plate, a 384 well plate, and a 1536 well plate.

9. The device of claim 1, wherein the device is a single use device.

10. The device of claim 1, wherein the device is a multiple use device.

11. A fluid exchange plug device configured for insertion into a petri dish, comprising:
a plug body having an upper surface and a lower surface;
two or more sockets positioned within the plug body, each socket having a first opening, a second opening and a lumen therebetween, wherein two or more sockets are accessible by the first opening on the upper surface of the plug body;
a stopper extending from the lower surface of the plug body having a first end and a second end, wherein the second end comprises at least one opening and wherein the stopper is sized to fit within a petri dish; and
wherein the second opening of each socket is fluidly connected to the at least one opening of the second end by at least one socket lumen extending through the plug body.

12. The fluid exchange plug device of claim 11, wherein the stopper further comprises a middle section positioned anywhere along the length of the stopper, and wherein the middle section has a diameter smaller than a diameter of the stopper.

13. The device of claim 12, wherein the stopper comprises a gasket or O-ring positioned on the middle section.

14. The device of claim 11, wherein the stopper extends from the lower surface of the plug body by a height ranging between about 1-40 mm.

15. The device of claim 11, wherein the device further comprising at least one channel embedded in the second end of the stopper.

16. The device of claim 15, wherein the stopper is sized to fit flush against a lower surface of a petri dish.

17. The device of claim 11, wherein the two or more sockets each comprise a threaded interior.

18. The device of claim 11, wherein the two or more sockets each are sized to receive fluidic or microfluidic tubing.

19. The device of claim 11, wherein the device is a single use device.

20. The device of claim 11, wherein the device is a multiple use device.
